(19) United States
(12) Patent Application Publication
(10) Pub. No.: US 2016/0089102 A1
(43) Pub. Date: Mar. 31, 2016

(54) COMPACT MEDICAL X-RAY IMAGING APPARATUS

(71) Applicant: TSUKUBA TECHNOLOGY CO., LTD., Tsukuba-shi (JP)

(72) Inventors: Bo Wang, Tsukuba-shi (JP); Norio Saito, Tsukuba-shi (JP); Xiaojun Liu, Tsukuba-shi (JP); Ryoichi Suzuki, Tsukuba-shi (JP)

(73) Assignee: TSUKUBA TECHNOLOGY CO., LTD., Tsukuba-shi (JP)

(21) Appl. No.: 14/964,283

(22) Filed: Dec. 9, 2015

Related U.S. Application Data
(63) Continuation of application No. PCT/JP2014/067825, filed on Jul. 3, 2014.

(30) Foreign Application Priority Data
Jul. 3, 2013 (JP) 2013-140230

(51) Int. Cl. A61B 6/00; H01J 35/06

(52) U.S. Cl. CPC A61B 6/542; A61B 6/4405; H01J 35/065; A61B 6/56; H01J 2201/30469; H01J 2235/062

(57) ABSTRACT

The present invention provides a compact medical X-ray imaging apparatus, which is a portable X-ray imaging apparatus capable of capturing clear X-ray images while maintaining low radiation exposure. The compact medical X-ray imaging apparatus comprises of: a carbon nanostructure triode cold cathode X-ray tube that radiates X-rays; an X-ray image sensor that captures an image of X-rays that pass through a patient; The first detector that detects the X-ray radiation dose and is positioned between the carbon nanostructure triode cold cathode X-ray tube and the X-ray image sensor, while out of the X-ray irradiation area for the imaging sensor; the second detector that detects the X-ray dose and is positioned in the center on one side of the X-ray image sensor frame; the third detector that detects the X-ray dose and is positioned on the other side of the X-ray image sensor frame facing to the second detector with the detection surface of the image sensor in between the second and third detector; a power supply which supplies a negative and a positive high-voltage pulse to the cathode and anode of the carbon nanostructure triode cold cathode X-ray tube respectively; and an X-ray imaging controller which acquires detection data from the first detector, second detector and third detector in addition to the distance from the carbon nanostructure triode cold cathode X-ray tube to the X-ray image sensor, calculates the X-ray radiation dose and amount of decay, determines the optimum X-ray dose for the patient and the voltage of the carbon nanostructure triode cold cathode X-ray tube, controls the pulse number and pulse width of the high-voltage pulse of the carbon nanostructure triode cold cathode X-ray tube, as well as the voltage of the cathode and the anode with feedback control means.

### 1. TECHNICAL FIELD

The present invention relates to a portable compact medical X-ray imaging apparatus, which can capture clear X-ray images while maintaining lower radiation exposure, possible to increase the service life of X-ray sources.

### 2. DESCRIPTION OF RELATED TECHNOLOGY

As a portable compact medical X-ray imaging apparatus, multiple patent documents from 1 to 8 are disclosed. For example, in accordance with patent document 4, a cold cathode electron source is taken as an X-ray source to achieve miniature; in accordance with non-patent document 1 and patent document 10, a cold cathode electron source is also disclosed; in accordance with patent document 9, a technology associated with the long service life of a cold cathode is disclosed.

### CITATION LIST

### Patent Literature

Patent Document 1: JP-A-2012-95715
Patent Document 2: JP-A-2012-70885
Patent Document 3: JP-A-2012-20835
Patent Document 4: JP-A-2012-65769
Patent Document 5: JP-A-2012-65768
Patent Document 6: JP-A-2012-56170
Patent Document 7: JP-A-2011-251005
Patent Document 8: JP-A-2011-253727
Patent Document 9: JP-A-2011-181276
Patent Document 10: JP-A-2012-133897

### Non Patent Literature

Non Patent Document 1: http://beam-physics.kek.jp/bpc/procs/suzuki.pdf

Subminiature electron accelerator driven by dry battery and development and application of high-energy X-ray source published on Mar. 29th, 2009 and invented by Ryouichi Suzuki in National Institute of Advanced Industrial Science and Technology.

### 3. SUMMARY OF INVENTION

### Technical Problem

None portable compact medical X-ray imaging apparatus does consider the optimum X-ray dose possible to capture clear X-ray images while maintaining lower radiation exposure to patients, and the problem of the service life of an X-ray source. The X-ray source is degraded along with the usage of the cathode. Even if certain voltage is applied to the cathode, the preset X-ray radiation dose cannot be obtained. If this state exists, the X-ray source has to change.

Besides, in accordance with patent document 9, a method for increasing the service life of a cold cathode is disclosed. However, the emitter need more current to be activated.

In recent years, mini movable portable compact medical X-ray imaging apparatus that is applied to consultation for emergency treatments on disaster and accident, emergent diagnosis and home nursing attracts attention. Furthermore, it is desirable to maintain low radiation exposure to patients and capture clear images.

Therefore, the present invention aims at providing a portable compact medical X-ray imaging apparatus possible to capture the clear X-ray images while maintaining the low radiation exposure, and possible to increase the service life of X-ray sources.

### Solution to Problem

To solve the problem, the present invention provides the compact medical X-ray imaging apparatus with the following structures:

(1)

A compact medical X-ray imaging apparatus, which is a portable X-ray imaging apparatus capable of capturing clear X-ray images while maintaining low radiation exposure, wherein the compact medical X-ray imaging apparatus comprises:

a carbon nanostructure triode cold cathode X-ray tube that radiates X-rays;

an X-ray image sensor that captures an image of X-rays that pass through a patient;

a first detector that detects the X-ray radiation dose and that is positioned between the carbon nanostructure triode cold cathode X-ray tube and the X-ray image sensor, and within the range in which X-rays are irradiated rather than the X-ray effective imaging area irradiated by the X-ray image sensor;

a second detector that detects the X-ray dose and is positioned in the center part of one side of the frame of the X-ray image sensor;

a third detector that detects the X-ray dose and is positioned on one side face of the frame of the X-ray image sensor sandwiching the detection faces of the X-ray image sensor and facing the second detector;

a power supply which supplies a negative and a positive high-voltage pulse to the cathode and anode of the carbon nanostructure triode cold cathode X-ray tube respectively;

an X-ray imaging control device which acquires detection data from the first detector, second detector and third detector in addition to information concerning the distance from the carbon nanostructure triode cold cathode X-ray tube to the X-ray image sensor, calculates the X-ray radiation dose and amount of decay, determines the optimum X-ray dose for the patient and the voltage of the carbon nanostructure triode cold cathode X-ray tube, and provided with feedback control means that controls the pulse number and pulse width of the high-voltage pulse of the carbon nanostructure triode cold cathode X-ray tube, and the voltage of the cathode and the anode.

(2)

The compact medical X-ray imaging device according to (1), wherein

based on detection results of the first detector, the current decrement of the carbon nanostructure triode cold cathode X-ray tube in accompany with the degradation of the carbon nanostructure triode cold cathode X-ray tube is calculated; and the preset current value and X-ray dose of the carbon nanostructure triode cold cathode X-ray tube can stably generate for a long term by applying an additional voltage, which offsets the current decrement of the carbon nanostructure triode cold cathode X-ray tube, to the cathode side electrode of the carbon nanostructure triode cold cathode X-ray tube and reducing the additional voltage from the anode side voltage.

(3)

The compact medical X-ray imaging device according to (1) or (2), wherein

a detachable battery as an X-ray radiation unit power supply is disposed on an X-ray radiation unit.

(4)

The compact medical X-ray imaging device according to (1) to (3), wherein

the compact medical X-ray imaging device is provided with a retaining base; the retaining base comprises:

a base, on which an AC/DC adapter is disposed, and which is provided with a connecting wire and a plug for connecting the base to a commercial power supply;

a supporting arm that is vertically disposed on the base and is embedded into the X-ray radiation unit; and

a connector that is connected to the AC/DC adapter by leads and is disposed at the end part of the supporting arm;

the commercial power supply can also be supplied to the X-ray radiation part while the X-ray radiation unit is embedded into the connector and is retained.

(5)

The compact medical X-ray imaging device according to (4), wherein

the second connector that is connected to the X-ray image sensor, the second detector and the third detector is disposed on the retaining table, and the second connector is connected to the X-ray imaging control device by the leads disposed in the supporting arm.

(6)

The compact medical X-ray imaging device according to (4) or (5), wherein

a power supply change-over switch is disposed in the X-ray radiation unit and can select the commercial power supply or the battery to supply the power.

### Advantageous Effects of Invention

According to the structure of the present invention, the following effects are achieved: the carbon nanostructure triode cold cathode X-ray radiation tube is taken as a radiation source, so that the energy can be saved while an imaging part can be miniaturized. Furthermore, the X-ray source is integrated with the X-ray imaging control device and a power supply, so that the imaging apparatus can be movable.

The X-ray imaging control device is provided with feedback control means to reduce the radiation dose to the patient and also capture the clear X-ray images. Additionally, the X-ray source is degraded along with the usage of the carbon nanostructure triode cold cathode X-ray tube, and the decrement of the X-ray dose is compensated by increasing the applied voltage, so that the X-ray radiation dose of the carbon nanostructure triode cold cathode X-ray tube can be stabilized. Therefore, the service life of the carbon nanostructure triode cold cathode X-ray tube is increased, and the long service life becomes possible.

In addition, the AC/DC adapter is disposed on the retaining base, and a power supply inside a diagnosis room can correspond to achieve long service life.

### BRIEF DESCRIPTION OF DRAWINGS

FIG. 1 is an integrated structure of a compact medical X-ray imaging apparatus provided by the present invention and application of the compact medical X-ray imaging apparatus to capture X-ray images to the abdomen of a patient;

FIG. 2 is a plane graph of an X-ray image sensor of a compact medical X-ray imaging apparatus;

FIG. 3 is a schema diagram of a carbon nanostructure triode cold cathode X-ray tube;

FIG. 4 is a control block diagram of a compact medical X-ray imaging apparatus provided by the present invention;

FIG. 5 is a schema diagram of applying of the additional voltage to offset the decrement (degradation decrement) of the X-ray dose caused by degradation of the carbon nanostructure triode cold cathode X-ray tube;

FIG. 6 is a steric diagram of an X-ray radiation unit maintained on the retaining base and the front view of an operation panel of the X-ray radiation unit.

### LIST OF REFERENCE SIGNS

1: compact medical x-ray imaging apparatus;
2: X-ray image sensor;
2a: detection face;
2b: frame;
2c: handle;
2e: signal;
2f: X-ray detection apparatus set;
3: first detector;
3a: signal;
3b: second detector;
3c: signal;
3d: third detector;
3e: signal;
3f: hanger rod;
4: retaining base;
4a: base;
4b: upper arm;
4c: lower arm;
4d: connecting part;
4e: plug;
4f: leads;
4g: AC/DC adapter;
4h: connecting wire;
4i: second connector;
4k: outlet;
4m: wires;
5: X-ray radiation unit;
5a: carbon nanostructure triode cold cathode X-ray tube;
5b: cathode;
5c: anode;
5d: carbon nano cathode;
5e: electron;
5f: target;
5g: radiation opening;
5h: intermediate electrode;
5i: hole;
5k: grounding;
5m: X-ray;
5n: X-ray effective imaging area;
5o: outside the imaging area;
5p: shielding part;
5q: single three dry batteries;
6: X-ray imaging control device;
7: switch;
7a: communication;
7b: battery;
7c: power supply change-over switch;
8: PC;
8a: X-ray imaging software;
8c: control signal;
9: operation panel;
9a: power supply on/off switch;
9b: power supply light;
9c: liquid crystal display screen;
9d: imaged part setting button;
9e: body type setting button;
9f: X-ray image sensor setting button;
9g: confirmation button;
9h: negative direction movement button;
9i: positive direction movement button;
9k: X-ray radiation display light;
9m: external remote terminal;
9n: various function setting and selecting button;
9o: radiation time setting button;
9p: detector setting button;
9q: reset button;
9r: battery remained amount display;
10: patient

### 4. DESCRIPTION OF EMBODIMENTS

The following describes specific implementation manners of the present invention in details based on the drawings of the specification. However, the present invention is not limited by these specific implementation manners.

### Embodiment 1

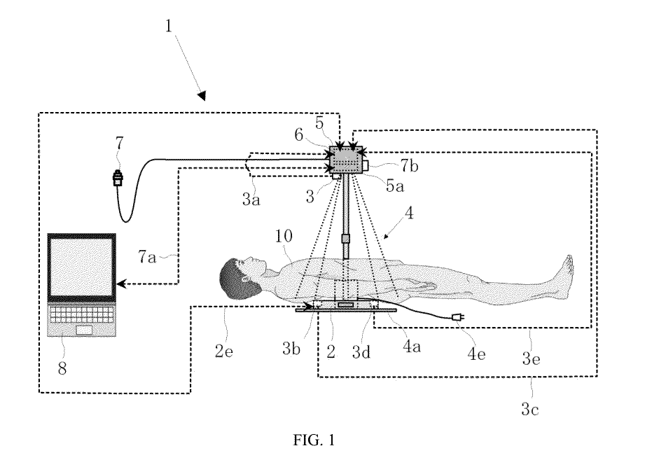

As shown in FIG. 1, a compact medical X-ray imaging apparatus 1 as one embodiment of the present invention comprises an X-ray image sensor 2, multiple detectors, a retaining base 4, an X-ray radiation unit 5, a power supply and a PC 8.

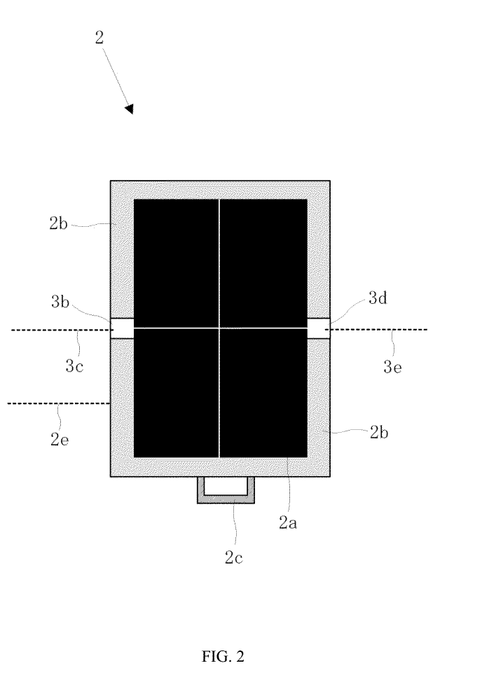

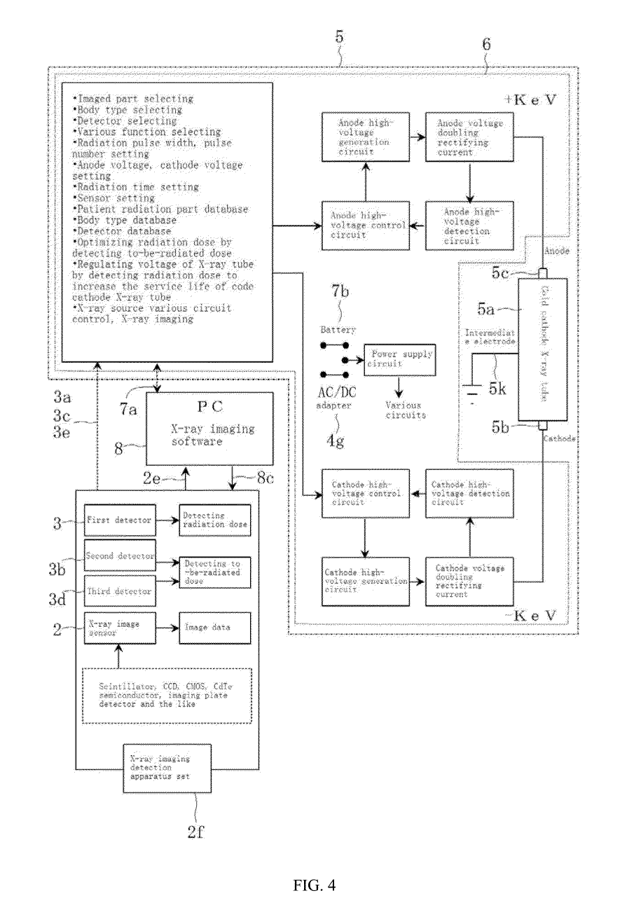

As shown in FIG. 1, FIG. 2 and FIG. 4, the X-ray image sensor 2 is disposed on a base 4a of the retaining base 4; a disease focus, which is captured by X-rays, of a patient is located above the X-ray image sensor 2; and the X-ray image sensor 2 detects X-rays that pass through the patient, obtains data that displays X-ray images and sends the data obtaining signal 2e to the X-ray imaging control device 6, and the PC 8 displays the X-ray images on the display based on the signal 2e. The X-ray image sensor 2 can be a scintillator, a CCD, a CMOS, a CdTe semiconductor, an imaging plate detector and the like.

As shown in FIG. 2, the X-ray image sensor 2 is provided with a detection face 2a that is disposed to detect X-rays that pass through the patient at the center, a frame 2b that surrounds the boundary of the X-ray image sensor 2, and a handle 2c that is disposed on the frame 2b to carry the X-ray image sensor 2. As shown in FIG. 2, a second detector 3b and a third detector 3d are exposed to the frame 2b.

As shown in FIG. 1 and FIG. 2, the multiple detectors consist of the first detector 3, the second detector 3b and the third detector 3d. The X-ray image sensor 2, the first detector 3, the second detector 3b and the third detector 3d form an X-ray detection apparatus set shown in FIG. 4.

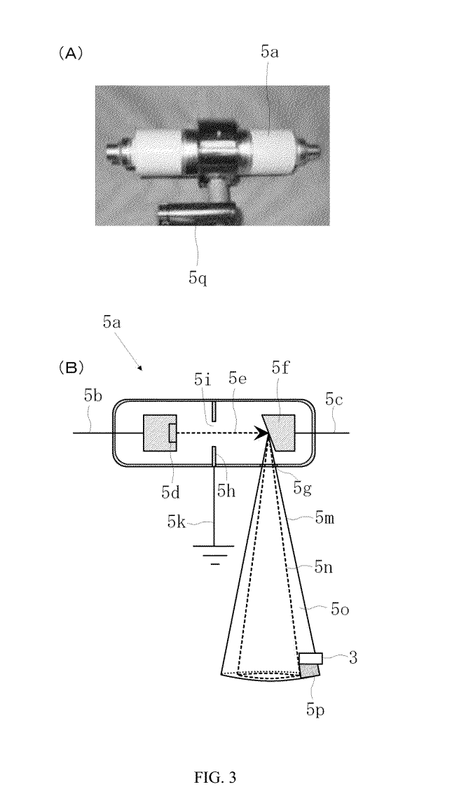

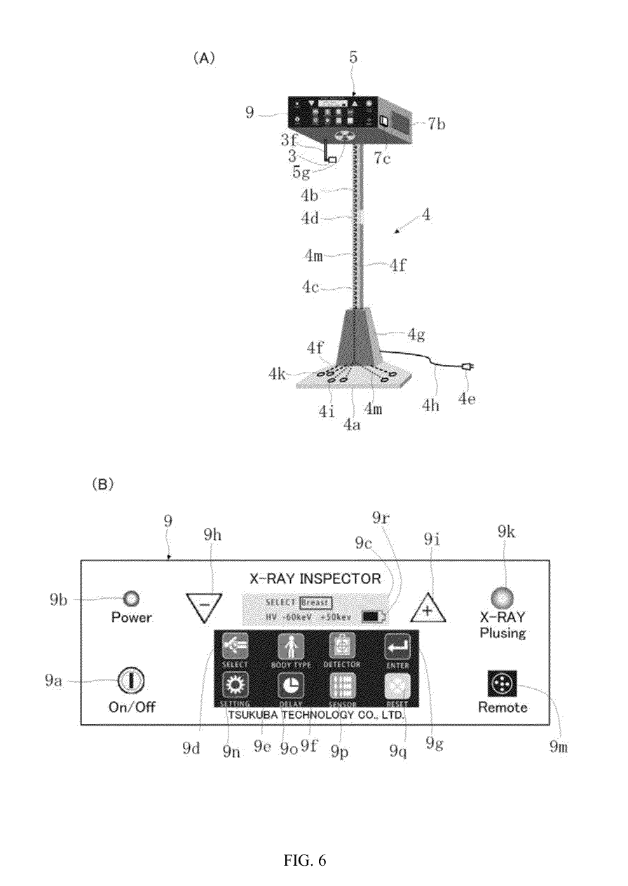

As shown in FIG. 1 and FIG. 3, the first detector 3 is disposed between the carbon nanostructure triode cold cathode X-ray tube 5a and the X-ray image sensor 2, and within the range 5m in which X-rays are irradiated outside the X-ray effective imaging region 5n irradiated by the X-ray image sensor 2, and the first detector 3 detects the X-ray radiation dose. The first detector 3 is disposed on the X-ray radiation unit 5, preferably at an appointed position. For example, the first detector 3 is connected to a rotatable supporting arm suspended on the X-ray radiation unit 5 (a hanger rod 3f in FIG. 6).

The second detector 3b is disposed at a central position of one side of the frame 2b of the X-ray image sensor 2 and is used for detecting the X-ray dose. The third detector 3d is positioned on other side of the frame 2b of the X-ray image sensor 2, in between the detection surface 2a of the X-ray image sensor 2 and the second detector 3b. Various detected data signals 3a, 3c, 3e are transmitted to the X-ray imaging control device 6 and are applied to the following information feedback control and X-ray dose stabilization control.

As shown in FIG. 1 and FIG. 6 (A), the retaining base 4 comprises a base 4a, a supporting arm that is vertically disposed on the base 4a and is embedded into the X-ray radiation unit 5 and a connector that is connected to an AC/DC adapter 4g by leads 4f and is disposed at the end part of the supporting arm, and the base 4a is provided with the AC/DC adapter 4g with a connecting wire 4h and a plug 4e which are connected to a commercial power supply. The supporting arm can be flexed or folded by connection part 4d, or consisted of an upper arm 4b and a lower arm 4c, and is very compact and high in portability. Due to the flexibility of the supporting arm, a detection unit can be disposed on the supporting arm and is used for detecting the distance between the X-ray radiation unit 5 and the X-ray image sensor 2. The detection unit can be a laser ranging device or a gear tester. Detection results are input into the X-ray imaging control device 6 and are applied to information feedback control.

The AC/DC adapter 4g of the base 4a is disposed at a position that can maintain the X-ray radiation part 5 considering the weight of the AC/DC adapter 4g. The leads 4f can be disposed inside the supporting arm, to further portability.

Furthermore, a second connector 4i is disposed on the base 4a of the retaining base 4 and is used for connecting the X-ray image sensor 2, the second detector 3b and the third detector 3d, and the second connector 4i are connected to the X-ray imaging control device 6 by wires 4m that are disposed in the supporting arm. Additionally, a socket 4k is disposed on the base 4a and is used for supplying power to the X-ray image sensor 2, the second detector 3b and the third detector 3d, and the socket 4k are connected to the commercial power supply by the AC/DC adapter 4g. Therefore, the compact medical X-ray imaging apparatus can be compactly assembled.

The X-ray radiation unit 5 is embedded into the end part (a connecting port) of the supporting arm, and the commercial power supply (in electrical connection) is supplied to the X-ray radiation unit 5 while retaining the X-ray radiation unit 5 to facilitate assembling. The X-ray radiation unit 5 can be preferably provided with a structure of the commercial power supply embedded in the supporting arm, and the X-ray radiation unit 5 can be further provided with a power supply change-over switch 7c to select a battery 7b or the commercial power supply (an AC/DC adapter) to supply power to select desired power supply. As shown in FIG. 6 (A), the power supply change-over switch 7c is disposed on the X-ray radiation unit 5.

As shown in FIG. 1 and FIG. 4, the X-ray radiation unit 5 consists of the carbon nanostructure triode cold cathode X-ray tube 5a and the X-ray imaging control device 6, and is commonly integrated with a detachable battery 7b to improve the portability. The X-ray imaging control device 6 can be disposed alone.

As shown in FIGS. 3 (A) and (B), the carbon nanostructure triode cold cathode X-ray tube 5a is mini-type, and electrons 5e generated by a carbon nano code cathode 5d on the side of a cathode 5b radiate a target 5f on the side of an anode 5c to generate X-rays 5m and emit out of a radiation opening 5g. The principles and structures of the electrons driven by a dry battery, a battery and a commercial power supply are described in patent document 10 and non patent document 1 in details. a power supply which supplies a negative and a positive high-voltage pulse to the cathode 5b and anode 5c of the carbon nanostructure triode cold cathode X-ray tube 5a respectively.

The X-ray imaging control device 6 obtains detection data of the first detector 3, the second detector 3b and the third detector 3d and the distance information between the carbon nanostructure triode cold cathode X-ray tube 5a and the X-ray image sensor 2 to calculate the X-ray radiation dose and amount of decay to determine the optimum X-ray dose to the patient 10 and the voltage of the carbon nanostructure triode cold cathode X-ray tube, and controls the pulse number and pulse width of the high-voltage pulse of the carbon nanostructure triode cold cathode X-ray tube 5a, and the voltage of the cathode 5b and the anode 5c, more specifically executes feedback control means shown in FIG. 4. Furthermore, the X-ray imaging control device 6 can perform various processes shown in FIG. 6 and stores various databases.

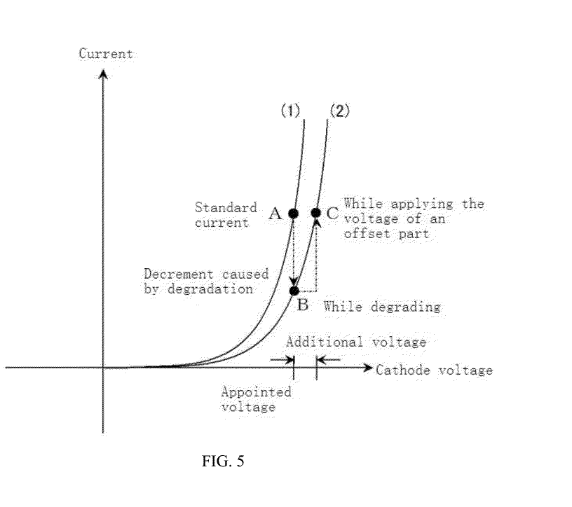

As shown in FIG. 5, to stabilize (increase the life of) the carbon nanostructure triode cold cathode X-ray tube 5a, based on detection results of the first detector 3, the current decrement of the carbon nanostructure triode cold cathode X-ray tube 5a accompanied with the deterioration of the carbon nanostructure triode cold cathode X-ray tube 5a is calculated, and the preset current value and X-ray dose of the carbon nanostructure triode cold cathode X-ray tube 5a can be stably generated for a long term by applying an additional voltage, which compensates the current decrement of the carbon nanostructure triode cold cathode X-ray tube 5a, to the cathode 5b side electrode of the carbon nanostructure triode cold cathode X-ray tube 5a reducing the additional voltage from the anode 5c side voltage. The result is shown in FIG. 5. The X-ray radiation dose from the carbon nanostructure triode cold cathode X-ray tube 5a is set to a preset value to increase the service life of the carbon nanostructure triode cold cathode X-ray tube 5a and improve the economic efficiency.

X-ray imaging starts after a switch 7 is switched on. After the switch 7 is switched on, the carbon nanostructure triode cold cathode X-ray tube 5a is electrified, and the feedback control means and other detectors are driven, so that optimum X-ray images are captured.

The PC 8 obtains data signals 2e, 3a, 3c, 3e detected and obtained by the X-ray image sensor 2, the first detector 3, the second detector 3b and the third detector 3d and transmits the data signals to the X-ray imaging control device 6. Additionally, the PC 8 can display X-ray images on a display while control the setting of the X-ray imaging control device 6. Communication 7a between the PC 8 and the X-ray imaging control device 6 can be wired communication or wireless communication.

A control recording mechanism such as a mini controller 6a is disposed on the X-ray radiation unit 5. As shown in FIG. 6, various buttons that set X-ray imaging conditions are disposed on an operation panel 9 of the X-ray radiation unit 5.

The following describes the buttons on the operation panel. A power supply on/off switch 9a is turned on or off to supply power to the X-ray imaging control device 6 or not. A power supply light 9b lights on when the power supply is switched on. A liquid crystal display screen 9c displays various sets and the battery remaining amount 9r. An imaged part setting button 9d records the voltage values of the anode side and the cathode side of the carbon nanostructure triode cold cathode X-ray tube corresponding to the optimum radiation dose of a representative imaged part.

A body type setting button 9e sets representative body type and can supplement and correct the voltage values of the imaged part. An X-ray image sensor setting button 9f can correct the characteristic difference of different X-ray image sensors. A radiation time setting button 90 is used for setting the radiation time to avoids unnecessary radiation for X-ray imaging operator. A detector setting button 9p can supplement and correct the characteristic difference of different detectors.

A negative direction movement button 9h and a positive direction movement button 9i can be used for selecting and switching the item contents of a text part on the liquid crystal display screen 9c with flashing. An X-ray radiation display light 9k is used for informing of the radiating of the X-rays, and is on while the X-rays irradiate. A confirmation button 9g is used for setting confirmation. A reset button 9q is used for setting reset. An external remote terminal 9m is a connector that is connected to the switch 7.

What is claimed is:

1. A compact medical X-ray imaging apparatus, which is a portable X-ray imaging apparatus capable of capturing clear X-ray images while maintaining low radiation exposure, wherein the compact medical X-ray imaging apparatus comprises:
a carbon nanostructure triode cold cathode X-ray tube that radiates X-rays;
an X-ray image sensor that captures an image of X-rays that pass through a patient;
a first detector that detects the X-ray radiation dose and that is positioned between the carbon nanostructure triode cold cathode X-ray tube and the X-ray image sensor, and within the range in which X-rays are irradiated rather than the X-ray effective imaging area irradiated by the X-ray image sensor;
a second detector that detects the X-ray dose and is positioned in the center part of one side of the frame of the X-ray image sensor;
a third detector that detects the X-ray dose and is positioned on one side face of the frame of the X-ray image sensor sandwiching the detection faces of the X-ray image sensor and facing the second detector;
a power supply which supplies a negative and a positive high-voltage pulse to the cathode and anode of the carbon nanostructure triode cold cathode X-ray tube respectively;
an X-ray imaging control device which acquires detection data from the first detector, second detector and third detector in addition to information concerning the distance from the carbon nanostructure triode cold cathode X-ray tube to the X-ray image sensor, calculates the X-ray radiation dose and amount of decay, determines the optimum X-ray dose for the patient and the voltage of the carbon nanostructure triode cold cathode X-ray tube, and provided with feedback control means that controls the pulse number and pulse width of the high-voltage pulse of the carbon nanostructure triode cold cathode X-ray tube, and the voltage of the cathode and the anode.

2. The compact medical X-ray imaging device according to claim 1, wherein
based on detection results of the first detector, the current decrement of the carbon nanostructure triode cold cathode X-ray tube in accompany with the degradation of the carbon nanostructure triode cold cathode X-ray tube is calculated; and
the preset current value and X-ray dose of the carbon nanostructure triode cold cathode X-ray tube can stably generate for a long term by applying an additional voltage, which offsets the current decrement of the carbon nanostructure triode cold cathode X-ray tube, to the cathode side electrode of the carbon nanostructure triode cold cathode X-ray tube and reducing the additional voltage from the anode side voltage.

3. The compact medical X-ray imaging device according to claim 1, wherein
an X-ray irradiation unit constituted by the carbon nanostructure triode cold cathode X-ray tube and the X-ray imaging control device,
a detachable battery as the X-ray radiation unit power supply is disposed on the X-ray radiation unit.

4. The compact medical X-ray imaging device according to claim 1, wherein
the compact medical X-ray imaging device is provided with a retaining base; the retaining base comprises:
a base, on which an AC/DC adapter is disposed, and which is provided with a connecting wire and a plug for connecting the base to a commercial power supply;
a supporting arm that is vertically disposed on the base and is embedded into the X-ray radiation unit; and
a connector that is connected to the AC/DC adapter by leads and is disposed at the end part of the supporting arm;
the commercial power supply can also be supplied to the X-ray radiation part while the X-ray radiation unit is embedded into the connector and is retained.

5. The compact medical X-ray imaging device according to claim 4, wherein
the second connector that is connected to the X-ray image sensor, the second detector and the third detector is disposed on the retaining table, and
the second connector is connected to the X-ray imaging control device by the leads disposed in the supporting arm.

6. The compact medical X-ray imaging device according to claim 4, wherein
a power supply change-over switch is disposed in the X-ray radiation unit and can select the commercial power supply or the battery to supply the power.
